(19) United States
(12) Patent Application Publication
(10) Pub. No.: US 2003/0040100 A1
(43) Pub. Date: Feb. 27, 2003

(54) Cell for the propagation of adenoviral vectors

(75) Inventors: Douglas E. Brough, Gaithersburg, MD (US); Imre Kovesdi, Rockville, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD

(21) Appl. No.: 09/911,020

(22) Filed: Jul. 23, 2001

(51) Int. Cl. C12N 7/02; C12N 5/02; C07H 21/04; C12N 5/00; C12N 7/01; C12N 7/00

(52) U.S. Cl. 435/235.1; 435/239; 435/325; 536/23.72; 536/24.1

(57) ABSTRACT

The invention provides a cell and a method of using the cell for the propagation of a replication-deficient adenoviral vector, wherein the cellular genome comprises a nucleic acid sequence whose expression produces a gene product that complements a replication-deficient adenoviral vector. The nucleic acid sequence is operatively linked to a chimeric expression control sequence comprising at least a functional portion of a CMV immediate early promoter/enhancer region and/or at least a functional portion of an adenoviral promoter, wherein the chimeric expression control sequence is upregulated by one or more viral proteins not produced by the nucleic acid sequence.

### FIELD OF THE INVENTION

This invention pertains to cells for the propagation of adenoviral vectors.

### BACKGROUND OF THE INVENTION

Recombinant eukaryotic viral vectors have become a preferred method of gene transfer for many researchers and clinicians. The human adenovirus is one of the most widely used recombinant viral vectors in current gene therapy protocols. As the use of adenoviral vectors becomes more prevalent, the need for systems that efficiently produce adenoviral vectors in a manner suitable for administration has increased.

A concern associated with recombinant adenoviral vectors is uncontrolled propagation of the vector upon administration. To address this concern, replication-deficient adenoviral vectors, typically lacking one or more regions of the adenoviral genome that are essential to replication, have been developed.

The production of replication-deficient adenoviral vectors is commonly accomplished by use of a complementing cell line, such as the 293 cell line developed by Graham et al. J. Gen. Virol., 36, 59-72 (1977), which provides the gene functions lacking in the replication-deficient adenoviral vector in trans. A problem associated with the 293 cell line is the possibility of homologous recombination between the replication-deficient adenoviral genome and the adenoviral genome portion of the complementation cell, resulting in production of replication-competent adenovirus (RCA). To reduce the frequency of RCA formation, several researchers have attempted to construct complementing cell lines that lack any homology to the adenoviral vector of interest (see, for example, International Patent Applications WO 94/28152 and WO 98/39411, and U.S. Pat. Nos. 5,994,128 and 6,033,908). Typically, such cell lines express gene functions associated with portions of the E1 and/or E4 regions of the adenoviral genome.

Construction of stable cell lines capable of such non-overlapping complementation has proven to be difficult. In particular, Gao et al., Human Gene Therapy, 11, 213-219 (2000), describes an A549 cell that stably expresses the E1 gene product from a promoter derived from the cytomegalovirus (CMV) immediate early (IE) promoter. The cell shares 612 nucleotides of homologous sequence with a standard E1-deleted Ad vector; however, the cell is unable to sustain replication of the E1-deleted vector. There are several reasons for the difficulty in generating stable complementing cell lines with reduced RCA occurrence. For example, such cell lines produce significant quantities of E1 and/or E4 gene products, resulting in undesired cytotoxic and/or cytostatic effects. High levels of E1A gene product expression, for example, induce apoptosis in host cells (Rao et al., PNAS, 89, 7742-7746 (1992)), while expression of E4 gene products induce p53-independent apoptosis in human cells (Marcellus et al., J. Virol., 72, 7144-53 (1998)). Thus, complementation cells, such as those known in the art, that constitutively express such factors may be associated with poor survival rates prior to and/or during adenoviral vector production.

Accordingly, there remains a need for alternative cells and methods for propagating replication-deficient adenoviral vectors. The invention provides such a cell and method. These and other advantages of the invention, as well as additional inventive features, will be apparent from the description of the invention provided herein.

### BRIEF SUMMARY OF THE INVENTION

The invention provides a cell for the propagation of a replication-deficient adenoviral vector having a cellular genome comprising a nucleic acid sequence, which upon expression produces a gene product that complements in trans for a deficiency in at least one essential gene function of an adenoviral genome. The nucleic acid sequence is operatively linked to a chimeric expression control sequence comprising at least a functional portion of a CMV immediate early promoter/enhancer region and at least a functional portion of an adenoviral promoter, wherein the chimeric expression control sequence is upregulated by one or more viral proteins not produced by the nucleic acid sequence.

In addition, the invention provides a method of propagating a replication-deficient adenoviral vector. The method comprises (a) providing a cell having a cellular genome comprising a nucleic acid sequence, which upon expression produces a gene product that complements in trans for a deficiency in at least one essential gene function of an adenoviral genome, and which is operatively linked to an expression control sequence that is upregulated by one or more adenoviral proteins not produced by the nucleic acid sequence, (b) introducing into the cell a replication-deficient adenoviral vector comprising an adenoviral genome deficient in the at least one essential gene function, and (c) maintaining the cell to propagate the replication-deficient adenoviral vector.

### DETAILED DESCRIPTION OF THE INVENTION

The invention provides a cell, for the propagation of a replication-deficient adenoviral vector, wherein the cell has a cellular genome comprising a nucleic acid sequence, which upon expression produces a gene product that complements in trans for a deficiency in at least one essential gene function of an adenoviral genome. The nucleic acid sequence is operatively linked to a chimeric expression control sequence comprising at least a functional portion of a CMV immediate early promoter/enhancer region, at least a functional portion of an adenoviral promoter, or both. The chimeric expression control sequence is upregulated by one or more adenoviral proteins not produced by the nucleic acid sequence. The cell desirably is suitable for the propagation (i.e., the replication of the entire life cycle, or the replication to any stage of the life cycle) of an adenoviral vector, more preferably a replication-deficient adenoviral vector.

The cell can be any suitable cell that comprises a genome capable of incorporating and preferably retaining the nucleic acid encoding a gene product that complements in trans for a deficiency in at least one essential gene function of an adenoviral genome. The cell desirably can propagate adenoviral vectors and/or adeno-associated viral (AAV) vectors when infected with such vectors or with nucleic acid sequences encoding the adenoviral or AAV genome. Most preferably, the cell can propagate a suitable replication-deficient adenoviral vector upon infection with an appropriate replication-deficient adenoviral vector or transfection with an appropriate replication-deficient viral genome.

Particularly desirable cell types are those that support high levels of adenovirus propagation. The cell preferably produces at least about 10,000 viral particles per cell and/or at least about 3,000 focus forming units (FFU) per cell. More preferably, the cell produces at least about 100,000 viral particles per cell and/or at least about 5,000 FFU per cell. Most preferably, the cell produces at least about 200,000 viral particles per cell and/or at least about 7,000 FFU per cell.

Preferably, the cell is, or is derived from, an anchorage dependent cell, but which has the capacity to grow in suspension cultures. Examples of suitable cells include human embryonic kidney (HEK) cells, human embryonic lung (HEL) cells, lung carcinoma cells, renal carcinoma cells, human retinal cells, human embryonic retinal (HER) cells, CHO cells, 786-0 cells, G-402 cells, ARPE-19 cells, KB cells, and Vero cells. Preferred cells are not, and/or are not derived from, HeLa cells. Preferred cells are, or are derived from, HER cells, HEK cells, and non-small cell lung carcinoma cells. Preferred HEK cells include cells derived from the 293 cell line (described in, e.g., Graham et al., supra), such as 293-ORF6 cells (described in, e.g., International Patent Application WO 95/34671 and Brough et al., J. Virol., 71, 9206-9213 (1997)). The non-small lung cell carcinoma cell can be a squamous/epidermoid carcinoma cell, an adenocarcinoma cell, or a large cell carcinoma cell. The adenocarcinoma cell can be an alveolar cell carcinoma cell or bronchiolo-alveolar adenocarcinoma cell. Preferred non-small cell lung carcinoma cells include A549 cells, alveolar cell carcinoma cells, and cells from cell lines derivative thereof. Other suitable non-small cell lung carcinoma cells include the cell lines NCI-H2126 (American Type Culture Collection (ATCC) No. CCL-256), NCI-H23 (ATCC No. CRL-5800), NCI-H1299 (ATCC No. CRL-5803), NCI-H322 (ATCC No. CRL-5806), NCI-H358 (ATCC No. CRL-5807), NCI-H810 (ATCC No. CRL-5816), NCI-H1155 (ATCC No. CRL-5818), NCI-H647 (ATCC No. CRL-5834), NCI-H650 (ATCC No. CRL-5835), NCI-H1385 (ATCC No. CRL-5867), NCI-H1770 (ATCC No. CRL-5893), NCI-H1915 (ATCC No. CRL-5904), NCI-H520 (HTB-182), and NCI-H596 (ATCC No. HTB-178). Also suitable are squamous/epidermoid carcinoma lines that include HLF-a (ATCC No. CCL-199), NCI-H292 (ATCC No. CRL-1848), NCI-H226 (ATCC No. CRL-5826), Hs 284.Pe (ATCC No. CRL-7228), SK-MES-1 (ATCC No. HTB-58), and SW-900 (ATCC No. HTB-59), the large cell carcinoma line NCI-H661 (ATCC No. HTB-183), and the alveolar cell carcinoma line SW-1573 (ATCC No. CRL-2170).

The cell comprises at least one nucleic acid sequence as described herein, i.e., the cell can comprise one nucleic acid sequence as described herein or more than one nucleic acid sequence as described herein (i.e., two or more of the nucleic acid sequences). Such cell lines can be generated in accordance with standard molecular biological techniques as described in International Patent Application WO 95/34671 and U.S. Pat. No. 5,994,106. The nucleic acid sequence preferably is stably integrated into the nuclear genome of the cell. The nucleic acid sequence preferably is retained in the cellular genome (and the nucleic acid sequence, upon expression, preferably produces a gene product complementing in trans for a deficiency in at least one essential gene function of one or more regions of an adenoviral genome) for at least about 10, more preferably at least about 20, passages in culture (e.g., at least about 30, 40, 100, or more passages). Not to adhere to any particular theory, it is believed that genomic integration of the nucleic acid sequence encoding the complementing factor is required to generate stable cell lines for adenoviral vector production. Additionally, complementation by transient transfection employs both labor-intensive and inconsistent techniques, resulting in low adenovirus yield and difficulty associated with large-scale viral production. The introduction and stable integration of the nucleic acid into the genome of the cell requires standard molecular biology techniques that are well within the skill of the art, such as those described in Sambrook et al., Molecular Cloning, a Laboratory Manual, 2d ed., Cold Spring Harbor Press, Cold Spring Harbor, N.Y. (1989), Watson et al., Recombinant DNA, 2d ed., Scientific American Books (1992), and Ausubel et al., Current Protocols in Molecular Biology, Wiley Interscience Publishers, NY (1995).

The “nucleic acid sequence” can be of any suitable source and/or synthetically prepared. The nucleic acid sequence can be obtained from, derived from, or based upon an adenoviral nucleic acid sequence. A sequence is “obtained” from a source when it is isolated from that source. A sequence is “derived” from a source when it is isolated from a source but modified in any suitable manner (e.g., by deletion, substitution (mutation), insertion, or other modification to the sequence) so as not to disrupt the normal function of the source gene. A nucleic acid sequence is “based upon” a source when the sequence is a sequence more than about 70% homologous (preferably more than about 80% homologous, more preferably more than about 90% homologous, and most preferably more than about 95% homologous) to the source but obtained through synthetic procedures (e.g., polynucleotide synthesis, directed evolution, etc.). Identifying such homologous sequences can be accomplished using any suitable method, particularly through use of the GenBank sequence databases provided by the National Center for Biotechnology Information (NCBI). Determining the degree of homology, including the possibility for gaps, can be accomplished using any suitable method (e.g., BLASTnr, provided by GenBank).

The nucleic acid sequence can be obtained or derived from the same or different serotype of adenovirus as the adenoviral vector to be propagated in the cell. The nucleic acid sequence and the adenoviral vector preferably are obtained from a group C adenovirus, more preferably from a serotype 2 or 5 adenovirus. Moreover, the nucleic acid sequence can include one or more mutations (e.g., point mutations, deletions, insertions, etc.) from the corresponding naturally occurring adenoviral coding sequence. Thus, where mutations are introduced in the nucleic acid sequence to effect one or more amino acid substitutions in an encoded gene product, such mutations desirably effect such amino acid substitutions whereby codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, codons encoding neutral non-polar residues (A, F, I, L, M, P, V, and W) are substituted with codons encoding neutral non-polar residues.

The nucleic acid sequence can be any suitable nucleic acid sequence as described herein that, upon expression, produces one or more gene products that complement for one or more deficiencies in any adenoviral essential gene functions (i.e., functions necessary for adenovirus propagation). By “complements for a deficiency in an essential gene function of an adenoviral genome” is meant that the gene product encoded by the adenoviral nucleic acid sequence exhibits an adenoviral gene function that is essential (i.e., necessary) for an adenoviral vector to propagate in a cell. For example, the gene product can induce transcription of promoters regulated by the E1 A protein, such as the E2A promoter.

The gene product encoded by the adenoviral nucleic acid sequence can be an RNA sequence or a protein (e.g., a peptide or a polypeptide). Preferably, the gene product encoded by the adenoviral nucleic acid sequence is a protein. Typically, and preferably, the nucleic acid will, upon expression, produce an adenoviral protein that provides an essential gene function. Examples of such proteins include the proteins of the E1A region, the E1B region, the E2 region (particularly the adenoviral DNA polymerase and terminal protein), the E4 region (particularly the protein encoded by open reading frame (ORF) 6 of the E4 region), the L1-L5 regions, and the IVa2 region of the adenoviral genome. The nucleic acid sequence also can encode the VAI or VAII regions of the adenoviral genome.

The nucleic acid sequence, upon expression, produces at least one gene product that provides an adenoviral essential gene function, i.e., that complements in trans for one or more deficiencies in any adenoviral essential gene function (i.e., a function that is necessary for adenovirus propagation). The nucleic acid sequence, upon expression, can produce a gene product that complements for two or more deficiencies in adenoviral essential gene functions (from the same or different regions of the adenoviral genome). The nucleic acid sequence, upon expression, can produce two or more gene products, each of which complements for a deficiency (i.e., at least one deficiency, including but not limited to, two or more deficiencies) in adenoviral essential gene functions (from the same or different regions of the adenoviral genome).

Essential adenoviral gene functions are those gene functions that are required for propagation (i.e., replication) of a replication-deficient adenoviral vector. Essential gene functions are encoded by, for example, the adenoviral early regions (e.g., the E1, E2, and E4 regions), late regions (e.g., the L1-L5 regions), and genes involved in viral packaging (e.g., the IVa2 and pIX genes). Thus, the gene product encoded by the nucleic acid sequence complements for a deficiency in at least one adenoviral essential gene function encoded by the early regions, late regions, viral packaging regions, or combinations thereof, including all adenoviral functions (e.g., to enable propagation of adenoviral amplicons comprising only inverted terminal repeats (ITRs) and the packaging signal or only ITRs and an adenoviral promoter).

The gene product desirably complements for a deficiency in at least one essential gene function of one or more regions of the adenoviral genome selected from the early regions, e.g., the E1, E2, and E4 regions. Preferably, the gene product complements in trans for a deficiency in at least one essential gene function of the E1 region of the adenoviral genome. More preferably, the gene product complements in trans for a deficiency in at least one essential gene function of an adenoviral E1A coding sequence and/or an adenoviral E1B coding sequence (which together comprise the E1 region). In that respect, one gene product can complement in trans for a deficiency in at least one essential gene function of the E1A coding sequence and another (i.e., different) gene product can complement in trans for a deficiency in at least one essential gene function of the E1B coding sequence. In addition or alternatively to the gene product(s) complementing in trans for the aforementioned deficiencies in adenoviral essential gene functions, the same or different gene product(s) can complement for a deficiency in at least one essential gene function of the E2 (particularly the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome.

Although not preferred, a helper virus can be provided to the cell in the event that the cell does not complement for all deficiencies in essential gene functions of the adenoviral genome of the adenoviral vector to be propagated. The helper virus contains coding sequences that, upon expression, produce gene products which provide in trans those gene functions that are necessary for adenoviral propagation (e.g., the pIX gene function). In other words, the helper virus can comprise any adenoviral nucleic acid sequence that is not required in cis (e.g., the ITRs and packaging signal) for propagation.

The cell can further comprise an “enhancing” nucleic acid sequence which upon expression produces at least one gene product that enhances propagation of a replication-deficient adenoviral vector without necessarily complementing for a deficiency in an adenoviral essential gene function, so as to propagate more replication-deficient adenoviral vectors when present in the cell than when the “enhancing” nucleic acid sequence is absent from the cell. Although genomic integration of this “enhancing” nucleic acid sequence is preferred, the “enhancing” nucleic acid sequence also can be maintained in the cell extrachromosomally (e.g., on a plasmid).

The “enhancing” nucleic acid sequence can be an adenoviral nucleic acid sequence that encodes at least one adenoviral gene product. In particular, the adenoviral gene product can be a protein encoded by, for example, the E1, E2, or E4 regions. The adenoviral gene product also can be a protein encoded by the late regions of the adenoviral genome, such as those encoded by the L1-L5 regions. Alternatively, the “enhancing” nucleic acid sequence can encode the adenoviral IVa2 protein, the pIX protein, or virus-associated RNA (e.g., VA-RNA I or II). The “enhancing” nucleic acid sequence also can be an animal or non-adenoviral nucleic acid sequence. The “enhancing” nucleic acid sequence can encode, for example, an animal protein that inhibits and/or prevents apoptosis (e.g., Bc1-2). Moreover, the “enhancing” nucleic acid sequence can encode, for example, an RNA molecule or protein that improves the efficiency or rate of replication-deficient adenoviral vector propagation.

The nucleic acid sequence encoding an gene product is operatively linked to a chimeric expression control sequence that is necessary for expression of the nucleic acid sequence to produce the gene product. An “expression control sequence” is any nucleic acid sequence that promotes, enhances, or controls expression (typically and preferably transcription) of another nucleic acid sequence. Typically and preferably, the expression control sequence comprises double-stranded DNA. Alternatively, the expression control sequence comprises double-stranded RNA, an RNA-DNA hybrid, or synthetically generated nucleotides. Typically and preferably, the expression control sequence comprises or consists essentially of a nucleic acid sequence that functions to direct the binding of RNA polymerase and thereby promotes transcription of the operatively linked nucleic acid sequence (e.g., a promoter sequence or portion thereof). A nucleic acid sequence is “operatively linked” to an expression control sequence when the expression control sequence is capable of promoting, enhancing, or controlling expression (typically and preferably transcription) of that nucleic acid sequence.

The expression control sequence is chimeric in that it comprises at least two nucleic acid sequence portions obtained from, derived from, or based upon at least two different sources (e.g., two different regions of an organism's genome, two different organisms, or an organism combined with a synthetic sequence). A sequence is “obtained” from a source when it is isolated from that source. A sequence is “derived” from a source when it comprises a sequence isolated from a source but modified in any suitable manner (e.g., by deletion, substitution (mutation), or other modification to the sequence). A sequence is “based upon” a source when the sequence is a sequence highly homologous to the source but obtained through synthetic procedures (e.g., polynucleotide synthesis, directed evolution, etc.). Preferably, the two different nucleic acid sequence portions exhibit less than about 40%, more preferably less than about 25%, and even more preferably less than about 10% nucleic acid sequence identity to one another (which can be determined by methods described elsewhere herein). Typically, the chimeric expression control sequence will comprise expression control sequences obtained or derived from at least two different eukaryotic viruses, preferably wherein only one of the expression control sequences is obtained from, derived from, or based upon the expression control sequence of an adenovirus. Preferably, the non-adenovirus portion of the expression control sequence is obtained or derived from a eukaryotic virus capable of infecting mammals, preferably humans, and desirably comprises a DNA genome, more desirably a double stranded DNA genome.

The chimeric expression control sequence can comprise any suitable type of individual expression control sequences, including promoters, enhancers, other regulatory sequences, or portions thereof (e.g., a Kozak consensus sequence, TATA box, or other DNA binding protein recognized sequence). The chimeric expression control sequence can comprise additional sequences which can exhibit expression control sequence activity alone or in conjunction with the other portions of the chimeric expression control sequence. Those additional sequences can be derived or obtained from additional sources (e.g., a third heterologous sequence obtained from a different source than the first and second sequences that are part of the chimeric expression control sequence). Preferably, the chimeric expression control sequence comprises a functional portion of a first sequence, which operates as an enhancer in the chimeric expression control sequence, from a first source, and a functional portion of a second sequence which operates as a promoter, from a second source.

In accordance with the invention, a “functional portion” is any portion of an expression control sequence that measurably promotes, enhances, or controls expression (typically transcription) of an operatively linked nucleic acid. Such regulation of expression can be measured via RNA or protein detection by any suitable technique, and several such techniques are known in the art. Examples of such techniques include Northern analysis (see, e.g., Sambrook et al., supra, and McMaster and Carmichael, PNAS, 74, 4835-4838 (1977)), RT-PCR (see, e.g., U.S. Pat. No. 5,601,820, and Zaheer et al., Neurochem Res., 20, 1457-1463 (1995)), in situ hybridization methods (see, e.g., U.S. Pat. Nos. 5,750,340 and 5,506,098), antibody-mediated techniques (see, e.g., U.S. Pat. Nos. 4,367,110, 4,452,901, and 6,054,467), and promoter assays utilizing reporter gene systems such as the luciferase gene (see, e.g., Taira et al., Gene, 263, 285-292 (2001)). Eukaryotic expression systems in general are further detailed in Sambrook et al., supra.

An enhancer is any cis-acting polynucleotide sequence that promotes, induces, or otherwise controls (e.g., inhibits) expression (preferably transcription) of one or more operatively linked nucleic acid sequences. An enhancer that inhibits transcription also is termed a “silencer.” The enhancer can function in either a direct or reverse orientation with respect to the nucleic acid sequence (e.g., from a position “downstream” of the operatively linked nucleic acid sequence) and over a relatively large distance (e.g., several kilobases (kb)) from an operatively linked nucleic acid sequence. Accordingly, the enhancer can be any nucleic acid sequence that can function to induce, promote, or control expression in either orientation and/or at various distances from the operatively linked nucleic acid sequence. In contrast, a promoter will operate in a sequence specific manner, typically in the same orientation with and upstream from the nucleic acid sequence, and in a more localized manner. For example, most eukaryotic promoters recognized by RNA polymerase II have a TATA box that is centered around position 25-30 upstream of the transcription start site and has the consensus sequence TATAAAA. Several promoters have a CAAT box around position 90 with the consensus sequence GGCCAATCT. Typically, the promoter will exhibit greater control over the operatively linked nucleic acid sequence. Thus, the promoter can be any nucleic acid sequence which exhibits localized control over the operatively linked nucleic acid.

In a preferred embodiment of the invention, the chimeric expression control sequence comprises a non-adenoviral functional portion. Preferred functional portions of non-adenoviral expression control sequence portions in this respect are obtained or derived from a cytomegalovirus (CMV), preferably a human CMV, and more particularly from the human CMV immediate early (IE) promoter/enhancer region. Advantageously, the CMV IE portion exhibits an enhancer activity in the chimeric expression control sequence. Enhancer activity can be determined by any suitable method, such as an enhancer trap as described in Boshart et al., Cell, 41, 521-530 (1985). Preferably, the CMV IE enhancer portion exhibits upregulation of operatively linked adenoviral genes in the presence of an adenoviral protein not expressed by the cellular genome and/or adenoviral vectors which lack adenoviral vector proteins expressed by the cellular genome. The CMV IE enhancer portion can be of any suitable size and comprise any suitable sequence derived from, based upon, or obtained from the wild-type CMV IE promoter/enhancer sequence (as described in Thomsen et al., PNAS, 81, 659-663 (1984), Jahn et al., J. Virol., 49, 363-370 (1984), Jahn et al., In: Herpseviruses, F. Rapp, ed., Alan Liss, Inc., New York, pp. 455-463 (1984), and Boshart et al., Cell, 41, 521-530 (1985)). Preferably, the CMV IE enhancer comprises a nucleic acid sequence which exhibits at least about 75%, desirably at least about 85%, and more preferably at least about 95% nucleic acid sequence identity to (e.g., at least 97% identity to, or 100% identical with) SEQ ID NO: 1. However, the invention is not limited to this exemplary sequence. Indeed, genetic sequences can vary between different strains, and this natural scope of allelic variation is included within the scope of the invention. Determining the degree of homology, including the possibility for gaps, can be accomplished using any suitable method (e.g., BLASTnr, provided by GenBank).

Additionally and alternatively, the CMV IE enhancer desirably includes any sequence that hybridizes to SEQ ID NO: 1 under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., supra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at (i) 42° C. in 0.2×SSC, (ii) at 55° C. in 50% formamide and (iii) at 55° C. in 0.1×SSC (preferably in combination with EDTA). Additional details and explanation of stringency of hybridization reactions are provided in, e.g., Ausubel et al., supra.

Preferably, a portion of the chimeric expression control sequence is obtained or derived from an adenovirus, and optionally (though not necessarily) from an adenovirus of the same serotype as the adenovirus from which the nucleic acid encoding the adenoviral protein is obtained from, derived from, or based upon, when the nucleic acid sequence is obtained from, derived from, or based upon an adenovirus. Preferred adenovirus genomes are obtained, derived, or based upon Group C adenoviruses and more preferably are obtained or derived from a serotype 2 or serotype 5 adenovirus. By incorporation of an adenoviral expression control sequence portion into the chimeric expression control sequence, stable cell lines are more readily obtained (as described further herein). The presence of the adenoviral expression control sequence portion is further believed to provide better control over expression of the operatively linked adenoviral-protein-encoding gene.

The chimeric expression control sequence can exhibit any suitable type of expression control sequence activity. Preferably, the chimeric expression control sequence exhibits a promoter activity. Preferably, the chimeric expression control sequence comprises an adenoviral promoter TATA box-associated sequence. A TATA-box associated sequence can be any sequence comprising the consensus sequence TATA (SEQ ID NO: 2), preferably positioned about 20-30 nucleotides upstream of a protein-encoding gene's transcription start site, which directs RNA polymerase binding and transcription of the operatively linked nucleic acid sequence. Desirably, the adenoviral TATA-box associated sequence promotes the production of stable cell lines compared to cell lines comprising only a heterologous promoter/enhancer region (e.g., a cell line which is capable of survival and adenoviral production after at least about 3, more preferably at least about 5, even more preferably at least about 10, advantageously at least about 20, and optimally at least about 100 passages). The adenoviral promoter TATA box-associated sequence can be obtained from any suitable adenoviral promoter. The TATA box-associated sequence can be of any suitable length. Typically, the adenoviral TATA box-associated sequence will be 135 nucleotides in length.

E1A TATA box-associated sequences are particularly preferred in cells of the invention in which the adenoviral promoter portion is linked to a portion of the E1A region of the adenoviral genome. Desirably, the E1A TATA box-associated sequence exhibits at least about 75% nucleic acid sequence identity, more preferably at least about 80% sequence identity, even more preferably at least about 90% nucleic acid sequence identity, and optimally at least about 95% sequence identity to (e.g., at least 97% identity to, or 100% identical with) SEQ ID NO: 3. However, the invention is not limited to this exemplary sequence. Indeed, genetic sequences can vary between different strains, and this natural scope of allelic variation is included within the scope of the invention. Additionally and alternatively, the E1A TATA box-associated sequence can include any sequence that hybridizes to SEQ ID NO: 3 under at least moderate, preferably high, stringency conditions. Determining the degree of homology and performing nucleic acid hybridizations can be accomplished using the methods discussed herein or any other suitable method.

The chimeric expression control sequence can be generated using standard molecular biology techniques, such as those described in Sambrook et al., supra. The chimeric expression control sequence can be inserted in the cellular genome using any suitable technique, such as, for example, those described in Sambrook et al., supra. Suitable transfection methods include but are not limited to calcium phosphate or DEAE-dextram-medicated transfection, polybrene transfection, protoplast fusion, electroporation, liposome-mediated transfection, or direct microinjection of DNA.

The function of the chimeric expression control sequence is induced, promoted, or enhanced (i.e., “upregulated”) by the presence of one or more adenoviral proteins not produced by the nucleic acid sequence of the inventive cell, typically and preferably resulting in inducing or promoting expression of the operatively linked adenoviral protein-encoding gene, as detected by standard methods such as those described herein.

The adenoviral protein(s) not produced by the nucleic acid sequence and that upregulates the chimeric expression control sequence can be any suitable protein obtained from, derived from, or based upon, a protein produced by an adenovirus. The precise adenoviral protein or combination of proteins will vary depending upon the components of the chimeric expression control sequence. Identification of suitable proteins can be determined by simple experimentation to determine whether administration or expression of the protein in the cell results in upregulation of the chimeric expression control sequence. Examples of suitable adenoviral proteins are discussed by Thomas Shenk, supra, and M. S. Horwitz, supra.

The one or more adenoviral proteins can be introduced to the cell independent from a viral particle or, typically and preferably, by their presence in or expression from a viral particle. The viral particle can be any suitable viral particle, including a non-intact viral particle (e.g., an incomplete viral particle) or a virus-like particle (VLP). Preferably, the viral particle is an intact adenoviral vector particle, and more preferably an intact replication-deficient adenoviral vector particle. The adenoviral vector particle can be a modified adenoviral vector particle, such as an adenoviral vector particle that exhibits a targeting function, such as the adenoviral vectors described in U.S. Pat. Nos. 5,559,099, 5,731,190, 5,712,136, 5,770,442, 5,846,782, 5,962,311, 5,965,541, and 6,057,155 and International Patent Applications WO 96/07734, WO 96/26281, WO 97/20051, WO 98/07865, WO 98/07877, WO 98/40509, WO 98/54346, and WO 00/15823. Other suitable adenoviral vectors are those comprising protein modifications that decrease the potential for immunological recognition by the host and resultant coat-protein directed neutralizing antibody production, as described in, e.g., International Patent Applications WO 98/40509 and WO 00/34496.

A suitable adenoviral protein can be introduced to the cell independent from a viral particle via expression of a nucleic acid encoding the protein delivered to the cell using vectors and transfection procedures in accordance with standard molecular biological techniques. Any suitable vector can be used for such a purpose. Suitable expression vectors are exemplified in Sambrook et al., supra, and can include a naked DNA or RNA vector (including, for example, a linear expression element or a plasmid vector such as pBR322, pUC 19/18, or pUC 118/119) or a precipitated nucleic acid vector construct (e.g., a CaPO₄ precipitated construct). The vector also can be a shuttle vector able to replicate and/or be expressed (desirably both) in both eukaryotic and prokaryotic hosts (e.g., a vector comprising an origin of replication recognized in both eukaryotes and prokaryotes). The vectors can be associated with salts, carriers (e.g., PEG), formulations which aid in transfection (e.g., sodium phosphate salts, Dextran carriers, iron oxide carriers, or gold bead carriers), and/or other pharmaceutically acceptable carriers, some of which are described herein. Alternatively or additionally, the vector can be associated with one or more transfection-facilitating molecules such as a liposome (preferably a cationic liposome), a transfection facilitating peptide or protein-complex (e.g., a poly(ethylenimine), polylysine, or viral protein-nucleic acid complex), a virosome, a modified cell or cell-like structure (e.g., a fusion cell), or a viral vector. Any suitable transfection method may be used to introduce the vector into the cell, such as, for example, those described in Sambrook et al., supra, and those described elsewhere herein.

The one or more adenoviral proteins can include one or more mutations (e.g., point mutations, deletions, insertions, etc.) from the corresponding naturally occurring adenoviral protein sequence. Thus, where mutations are introduced to substitute amino acid residues, positively-charged residues (H, K, and R) preferably are substituted with positively-charged residues; negatively-charged residues (D and E) preferably are substituted with negatively-charged residues; neutral polar residues (C, G, N, Q, S, T, and Y) preferably are substituted with neutral polar residues; and neutral non-polar residues (A, F, I, L, M, P, V, and W) preferably are substituted with neutral non-polar residues.

In an alternative embodiment, nucleic acid sequences encoding the one or more adenoviral proteins can reside in the cell episomally or as stable integrants in the cellular genome. In this context, the production of the one or more adenoviral proteins can be controlled by an inducible promoter system operatively linked to the nucleic acid sequences encoding the one or more adenoviral proteins.

The one or more adenoviral proteins not produced by the nucleic acid sequence of the inventive cell (e.g., the adenoviral vector particle comprising or expressing such a protein) can upregulate the chimeric expression control sequence in any suitable manner and to any suitable degree. For example, the presence of the one or more adenoviral proteins not produced by the nucleic acid sequence can result in an at least about a 10%, 20%, or 30% increase in the expression of the chimeric expression sequence-linked adenoviral coding sequence. Preferably, the presence of the adenoviral protein results in at least about a 40%, and more preferably at least about a 50%, increase (e.g., at least about a 60%, 70%, 100%, 200%, or even a 1,000-fold increase) in expression of a nucleic acid sequence operatively linked to the chimeric expression control sequence. The adenoviral protein can “induce” expression of the operatively linked nucleic acid sequence from non-detectable levels or merely enhance expression levels over “constitutive” expression levels. The adenoviral protein can upregulate the chimeric expression control sequence directly by, for example, physical interaction of the adenoviral penton protein with the chimeric expression control sequence. Alternatively, the adenoviral protein can upregulate the chimeric expression control sequence indirectly. For example, the adenoviral protein can interact with a molecule that represses expression from the chimeric expression control sequence. Such an interaction releases transcriptional repression by the molecule, resulting in upregulation of expression from the chimeric expression control sequence. Preferably, the chimeric expression control sequence is operatively linked to the adenoviral E1A gene. More preferably, the adenoviral protein induces E1A expression to levels sufficient to induce expression of the E1B protein. The degree and manner of upregulation associated with the chimeric expression control sequence can be determined by any suitable technique, such as the techniques described elsewhere herein or otherwise known in the art for measuring expression control sequence activity and/or gene expression.

The invention also provides a system comprising the inventive cell and a replication-deficient adenoviral vector comprising an adenoviral genome deficient in the at least one essential gene function (i.e., a replication-deficient adenoviral vector comprising the deficiencies complemented for by the inventive cell). The invention further provides a method of propagating a replication-deficient adenoviral vector. The method comprises (a) providing a cell having a cellular genome comprising a nucleic acid sequence, which upon expression produces a gene product (generally a protein) that complements in trans for a deficiency in at least one essential gene function of an adenoviral genome, and which is operatively linked to an expression control sequence that is upregulated by one or more adenoviral proteins not produced by the nucleic acid sequence, (b) introducing into the cell a replication-deficient adenoviral vector comprising an adenoviral genome deficient in the at least one essential gene function (i.e., a replication-deficient adenoviral vector comprising the deficiencies complemented for by the cell), and maintaining the cell to propagate the replication-deficient adenoviral vector (e.g., maintaining the cell under conditions suitable for adenoviral propagation, whereupon the adenoviral vector is propagated). Preferably, the expression control sequence is the chimeric expression control sequence described in the context of the inventive cell (i.e., preferably the cell utilized in the context of the inventive method is the inventive cell described herein).

The replication-deficient adenoviral vector preferably has an adenoviral genome deficient in at least one essential gene function of an early region of the adenoviral genome. In a preferred embodiment of the invention, the adenoviral vector comprises the one or more adenoviral proteins that upregulate the chimeric expression control sequence. Alternatively, the one or more adenoviral proteins that upregulate the chimeric expression control sequence can be introduced independently from the adenoviral vector. In still another alternative, the one or more adenoviral proteins can reside in the cell episomally or as stable integrants in the cellular genome. In this context, the expression of the one or more adenoviral proteins can be controlled by an inducible promoter system operatively linked to the nucleic acid sequences encoding the one or more adenoviral proteins. As will be appreciated by one of skill in the art, the one or more adenoviral proteins can be introduced to the cell using vectors and transfection procedures in accordance with standard molecular biological techniques discussed herein, as well as by providing the adenoviral proteins in the culture medium.

The adenoviral vector is deficient in at least one gene function (of the adenoviral genome) required for viral propagation (i.e., an adenoviral essential gene function), thereby resulting in a “replication-deficient” adenoviral vector. The adenoviral vector is deficient in the one or more adenoviral essential gene functions complemented for by the inventive cell to allow for propagation of the replication-deficient adenoviral vector when present in the cell.

Preferably, the adenoviral vector is deficient in at least one essential gene function of the E1 region, e.g., the E1 a region and/or the E1b region, of the adenoviral genome that is required for viral replication. The recombinant adenovirus also can have a mutation in the major late promoter (MLP), as discussed in International Patent Application WO 00/00628. More preferably, the vector is deficient in at least one essential gene function of the E1 region and at least part of the nonessential E3 region (e.g., an Xba I deletion of the E3 region). The adenoviral vector can be “multiply deficient,” meaning that the adenoviral vector is deficient in one or more essential gene functions in each of two or more regions of the adenoviral geonome. For example, the aforementioned E1-deficient or E1-, E3-deficient adenoviral vectors can be further deficient in at least one essential gene of the E4 region and/or at least one essential gene of the E2 region (e.g., the E2A region and/or E2B region). Adenoviral vectors deleted of the entire E4 region can elicit lower host immune responses. Examples of suitable adenoviral vectors include adenoviral vectors that lack (a) all or part of the E1 region and all or part of the E2 region, (b) all or part of the E1 region, all or part of the E2 region, and all or part of the E3 region, (c) all or part of the E1 region, all or part of the E2 region, all or part of the E3 region, and all or part of the E4 region, (d) at least part of the E1a region, at least part of the E1b region, at least part of the E2a region, and at least part of the E3 region, (e) at least part of the E1 region, at least part of the E3 region, and at least part of the E4 region, and (f) all essential adenoviral gene products (e.g., adenoviral amplicons comprising ITRs and the packaging signal only). The adenoviral vector can contain a wild type pIX gene. Alternatively, although not preferably, the adenoviral vector also can contain a pIX gene that has been modified by mutation, deletion, or any suitable DNA modification procedure.

The replication-deficient adenoviral vector can be generated by using any species, strain, subtype, or mixture of species, strains, or subtypes, of an adenovirus or a chimeric adenovirus as the source of vector DNA. The adenoviral vector can be any adenoviral vector capable of growth in a cell, which is in some significant part (although not necessarily substantially) derived from or based upon the genome of an adenovirus. The adenoviral vector preferably comprises an adenoviral genome of a wild-type adenovirus of group C, especially of serotype (i.e., Ad5). Adenoviral vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

The construction of adenoviral vectors is well understood in the art and involves the use of standard molecular biological techniques, such as those described in, for example, Sambrook et al., supra, Watson et al., supra, Ausubel et al., supra, and other references mentioned herein. Moreover, adenoviral vectors can be constructed and/or purified using the methods set forth, for example, in U.S. Pat. No. 5,965,358 and International Patent Applications WO 98/56937, WO 99/15686, and WO 99/54441.

When the cell is used to propagate a replication-deficient adenoviral vector, it is desirable to avoid a recombination event between the cellular genome (of the cell) and the adenoviral genome (of the adenoviral vector) that would result in the generation of a replication-competent adenovirus (RCA). As such, there is preferably insufficient overlap between the genome of the cell and the replication-deficient adenoviral vector genome to mediate a recombination event sufficient to result in a replication-competent adenovirus. If overlap exists, the overlapping sequences desirably are predominantly located in the nucleic acid flanking the coding region of the complementation factor (the “trans-complementing region”) in the cellular genome and the nucleotide sequences adjacent to the missing region(s) of the adenoviral genome. Ideally, there is no overlap between the cellular genome and the adenoviral vector genome. However, it is acceptable that partial overlap exists between the cellular genome and the adenoviral vector genome on one side of the trans-complementing region. In such an event, the region of homology preferably is contiguous with the trans-complementing region. For example, when the cell comprises a trans-complementing region comprising a nucleotide sequence of the adenoviral E1 region, the cell desirably lacks homologous sequences on the 5′ side (left side) of the trans-complementing region corresponding to the adenoviral inverted terminal repeats (ITRs) and packaging signal sequences, but contains homologous sequences on the 3′ side (right side) of the trans-complementing region. The region of homology is at least about 300 base pairs, preferably at least about 700 base pairs, more preferably at least about 1000 base pairs (e.g., at least about 1500 base pairs), and most preferably at least about 2000 base pairs.

The cell preferably is characterized by lacking the 5′ ITR, the packaging sequence, and the E1A enhancer of the adenoviral genome. The preferred cell is further characterized by desirably comprising the nucleic acid sequences encoding E1A, E1B, protein IX, and IVa2/partial E2B. In particular, the preferred cell comprises at least one adenoviral nucleic acid sequence which lacks nucleotides 1-361, yet comprises adenoviral nucleotides 3325-5708 located 3′ to the complementing region. Not to adhere to any particular theory, it is believed that a single recombination event in such a homologous region will not give rise to a replication competent adenoviral vector due to the absence of the 5′ ITR and packaging sequence. In a similar manner, a preferred cell that contains both the E1 and E4 regions sufficient to propagate E1-, E4-deleted adenoviral vectors can comprise a region of homology between the cellular genome and the adenoviral genome located 5′ or 3′ to the nucleic acid sequence encoding the E4 region.

The generation of RCA desirably is diminished such that (a) the cell produces less than about one replication-competent adenoviral vector for at least about 20 passages after infection with the adenoviral vector, (b) the cell produces less than about one replication-competent adenoviral vector in a period of about 36 hours post-infection, (c) the cell produces less than about one replication-competent adenoviral vector per 1×10¹⁰ total viral particles (preferably 1×10¹¹ total viral particles, more preferably 1×10¹² total viral particles, and most preferably 1×10¹³ total viral particles), or any combination of (a)-(c). Optimally, the amount of overlap between the cellular genome and the adenoviral genome (i.e., the genome of the adenoviral vector being propagated in the cell) is insufficient to mediate a homologous recombination event that results in a replication-competent adenoviral vector such that replication-competent adenoviruses are eliminated from the vector stocks resulting from propagation of the replication-deficient adenoviral vector in the cell. Virus growth yield and virus plaque formation have been previously described (see, e.g., Burlseson et al., Virology: a Laboratory Manual, Academic Press Inc. (1992)), and measuring RCA as a function of plaque forming units is described in U.S. Pat. No. 5,994,106.

The following examples further illustrate the invention but, of course, should not be construed as in any way limiting its scope.

### EXAMPLE 1

This example demonstrates the construction of a nucleic acid comprising an adenoviral E1 coding sequence.

An Ad2 E1 expression cassette was assembled in pKS (Stratagene) to generate pKSCMVE1. Initially, Ad2 nucleotides 362-917, including the E1A TATA box-associated sequence comprising nucleotides 362-497, was amplified by PCR, and the resulting Eco RI to C1a I fragment was cloned into pKS. Subsequently, the Ad2 sequences from 918-5708 were incorporated into pKS as a PCR-generated C1a I fragment. Finally, the CMV IE enhancer comprising nucleotides 174254-173843 of the human CMV genome was incorporated into pKS as a PCR-generated EcoR I fragment to generate pKSCMVE1. pZeoE1 (Invitrogen) was constructed by replacing the SV40 driven expression cassette with the E1 expression cassette from pKSCMVE1. Thus, pZeoE1 comprises the human CMV IE enhancer sequence, the Ad2 E1A TATA box-associated sequence comprising nucleotides 362-497, and the Ad2 E1A and E1B coding sequences.

The adenoviral vectors comprising coding sequences for B-glucoronidase, no transgene, β-galactosidase, TNF-α, secretory alkaline phosphatase, or vascular endothelial growth factor (VEGF) 121 (AdG, Adnull, AdZ, AdTNF, AdS, and AdVEGF121, respectively) under the control of a CMV IE promoter are known in the art. Each adenoviral vector comprises a deletion of nucleotides 356-3328 of the E1 region of the adenoviral genome.

The E1 expression cassette plasmid was tested for functionality by infection-transflection experiments in A549 cells. The cells were infected with AdG and thereafter transfected with pZeoE1. At 20 hours post-infection (h.p.i.) the amount of transgene expression (glucoronidase (gus) activity) was quantified, and vector DNA replication was detected by Southern blot analysis. The presence of E1 gene products expressed in trans from pZeoE1 increased transgene expression approximately 100-fold. The increase in gus activity was accompanied with adenovirus vector DNA replication. Thus, the expression cassette E1 gene products provided functional complementation.

The results of this example confirm the construction and proper functioning of a nucleic acid comprising a chimeric expression control sequence, (illustrated by a CMV IE enhancer, an E1 TATA-box associated sequence (Ad2 nucleotides 362-497), and the Ad2 E1A and E1B coding regions (nucleotides 498-5708)), which upon expression produces a gene product that complements in trans an adenoviral vector comprising a deficiency in at least one essential gene function (illustrated by a deficiency in the E1 region) of the adenoviral genome.

### EXAMPLE 2

This example demonstrates the construction of a complementing cell using the A549 cell line as the parent cell line.

A549 cells are continuous tumor cells derived from a human lung carcinoma with properties of type II alveolar epithelial cells (Lieber et al., Int. J Cancer, 17, 62-70, (1976)). A549 cells support productive wild-type adenovirus replication, and are adaptable to growth in serum free suspension culture (ATCC, CCL-185.1). A549 cells were transfected with linearized pZeoE1, placed under Zeocin selection, and resistant colonies were isolated. The cells were subsequently cultured using routine tissue culture techniques. Monolayers at passages 5 and 10 were screened for E1 complementation by a virus production assay (see, e.g., Burlseson et al., Virology: a Laboratory Manual, Academic Press Inc. (1992)). In that respect, cells were infected with Adnull at a multiplicity of infection (MOI) of 10, cell lysates were prepared at 3 days post-infection (d.p.i.), and the amount of active virus in the lysates was determined by a focal forming unit (FFU) assay (Cleghorn et al., Virology, 197, 564-575 (1993)). The detected yields of Adnull for each cell line at passages 5 and 10, which evidence the ability of the cell line to complement for an E1-deficiency in an adenoviral genome, are set forth in Table 1.

TABLE 1
Yield of Adnull from E1 cell lines at 3 d.p.i. (FFU/cell)*

| Cell | Passage | | Cell | Passage | | Cell | Passage | |
|---|---|---|---|---|---|---|---|---|
| line | 5 | 10 | line | 5 | 10 | line | 5 | 10 |
| P | 118 | T | 52 | 69 | T | R10 | 1681 | 65 |
| 1 | 136 | 53 | 53 | 454 | 250 | R11 | 111 | 12 |
| 2 | 256 | 45 | 55 | 36 | T | R13 | 134 | 0 |
| 3 | 153 | 0 | 57 | 39 | T | R14 | 133 | 26 |
| 5 | 42 | T | 61 | 108 | 25 | R15 | 63 | 0 |
| 9 | 179 | 21 | 63 | 141 | 25 | R24 | 125 | 125 |
| 11 | 20 | T | 70 | 133 | 0 | R26 | 175 | 150 |
| 16 | 25 | T | 71 | 75 | T | R29 | 650 | 900 |
| 18 | 80 | T | 75 | 46 | T | R36 | 212 | 120 |
| 21 | 218 | 57 | 76 | 8 | T | R38 | 25 | 12 |
| 24 | 42 | T | 77 | 166 | 42 | R50 | 62 | 26 |
| 25 | 830 | 700 | 80 | 311 | 0 | R52 | 83 | 12 |
| 28 | 280 | 46 | 81 | 79 | T | R57 | 37 | 35 |
| 31 | 218 | 10 | 85 | 358 | 12 | R59 | 40 | 23 |
| 32 | 410 | 360 | 88 | 51 | 0 | R60 | 55 | 28 |
| 33 | 275 | 54 | R1 | 47 | 21 | R65 | 37 | 25 |
| 46 | 708 | 550 | R9 | 55 | 45 | R66 | 125 | 142 |

*P = Zeocin-resistant cell line; the prefix ‘AB’ was added to the isolate number for the final name of each cell line; T = termination of the cell line due to problems with maintenance of the monolayer

As is apparent from the results set forth in Table 1, complementing cells of the invention were produced and confirmed to complement in trans an adenoviral genome comprising a deficiency in at least one essential gene function. In particular, AE25 and AE29 (lines 25 and R29 in Table 1, respectively) were the highest producing cell lines at passages 5 and 10 and formed high quality monolayers.

### EXAMPLE 3

This example demonstrates the ability of complementing cells of the invention to support viral replication and viral production.

AE25 and AE29 cells of Example 2 (lines 25 and R29 in Table 1, respectively) were infected with Adnull. The growth kinetics of Adnull in AE25 and AE29 cells were compared to the growth of the virus in 293 cells and A549 cells over a five-day time course. Virus growth yield, virus plaque formation, Southern blot, Northern blot, and PCR analyses have been previously described (see, e.g., Burlseson et al., supra, Sambrook et al., supra, and Innis et al., eds., PCR Protocols, A Guide to Methods and Applications, Academic Press, Inc. (1990)).

Adnull replication in AE25 cells had a one-day lag period compared to replication in 293 or AE29 cells. The overall yield of active particles in AE25 cells ranged from 25-50% of the yield from 293 cells. Adnull growth in AE29 cells did not show a lag period, and the number of active particles produced per cell was within 2-fold of 293 cells at 2 and 3 days post infection. Therefore, both AE25 and AE29 functionally complement for growth of E1 deleted virus to within at least about 50% of 293 cells. The results of these experiments are set forth in Table 2.

TABLE 2
Adnull growth kinetics in different host cells
(number of active particles produced per cell (pfu/cell))

Days post-infection

| cell line | 1 | 2 | 3 | 5 |
|---|---|---|---|---|
| 293 | 1000 | 4000 | 3000 | 2000 |
| AE25 | 0 | 550 | 700 | 1000 |
| AE29 | 160 | 2500 | 1500 | 850 |
| A549 | 0 | 0 | 0 | 0 |

The ability of AE25 and AE29 cells to support productive infection was measured by plaque formation assays. The plaque forming unit (pfu) titers of AdZ, AdTNF, and AdS were determined. The efficiency of plaque formation on AE25 and AE29 cells was about 5-60% of the best available plaque-forming 293 monolayer cell line. The results of these experiments are set forth in Table 3.

TABLE 3
Plaque formation efficiency of E1-complementing cell lines

| | Vector titer (pfu/ml) | | |
|---|---|---|---|
| Cell line | AdZ titer | AdTNF titer | AdS titer |
| 293 | 1.0 × 10¹⁰ | 2.0 × 10¹⁰ | 2.6 × 10⁹ |
| AE10 | 1.5 × 10⁸ | 1.0 × 10⁸ | 1.0 × 10⁸ |
| AE25 | 2.0 × 10⁹ | 2.5 × 10⁹ | 1.0 × 10⁹ |
| AE29 | 2.5 × 10⁹ | 2.0 × 10⁹ | 3.0 × 10⁹ |
| A549 | 0 | 0 | 0 |

The results of this example demonstrate the ability of A549-derived complementing cells (specifically, AE25 and AE29) to support the production of an E1-deleted Ad vector to within at least about 50% of the efficiency of 293 cells.

### EXAMPLE 4

This example demonstrates upregulation of the chimeric expression control sequence by adenoviral infection.

The number of copies of the E1 expression cassette integrated into the cellular genome of the AE25 and AE29 cells of Examples 2 and 3 was determined by Southern blot analysis. There was approximately one copy of the E1 expression cassette per cell integrated in both AE25 and AE29 cell lines. AE25 and AE29 cells were infected with an E1- and protein IX-deleted mutant adenoviral vector, H5dl313 (Jones and Shenk, Cell, 17, 683-89 (1979)). The expression of E1A and E1B adenoviral proteins in the AE25 and AE29 cells was induced 2-fold to 5-fold by infection with H5dl3 13 as compared to uninfected cells. Presumably, the upregulation of the E1A and E1B adenoviral proteins by infection occurred via activation of the chimeric CMV/adenovirus expression control sequence in the integrated E1 cassette.

This example confirms the upregulation of the chimeric expression control sequence by one or more adenoviral proteins not produced by the nucleic acid sequence that is incorporated into the cellular genome and provides, upon expression, the complementing function. Moreover, this example confirms that the adenoviral protein upregulating the chimeric control expression sequence can be provided by the replication-deficient adenoviral vector to be propagated in the cell line.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A cell for the propagation of a replication-deficient adenoviral vector having a cellular genome comprising a nucleic acid sequence, which upon expression produces a gene product that complements in trans for a deficiency in at least one essential gene function of an adenoviral genome, and which is operatively linked to a chimeric expression control sequence comprising at least a functional portion of a CMV immediate early promoter/enhancer region, at least a functional portion of an adenoviral promoter, or both, wherein the chimeric expression control sequence is upregulated by one or more adenoviral proteins not produced by the nucleic acid sequence.

2. The cell of claim 1, wherein the chimeric expression control sequence comprises at least a functional portion of a CMV immediate early promoter/enhancer region and at least a functional portion of an adenoviral promoter.

3. The cell of claim 1, wherein expression of the nucleic acid sequence complements in trans an adenoviral genome comprising deficiencies in at least one essential gene function of the E1 region of the adenoviral genome.

4. The cell of claim 3, wherein the nucleic acid sequence comprises an adenoviral E1A coding sequence and an adenoviral E1B coding sequence.

5. The cell of claim 4, wherein the cellular genome further comprises a nucleic acid sequence comprising an adenoviral E2 region, E4 region, or both.

6. The cell of claim 5, wherein the cellular genome comprises a nucleic acid sequence encoding an E4-ORF6 gene product.

7. The cell of claim 1, wherein the chimeric expression control sequence comprises a CMV immediate early enhancer.

8. The cell of claim 1, wherein the chimeric expression control sequence comprises an E1A TATA box-associated sequence.

9. The cell of claim 7, wherein the CMV immediate early enhancer comprises a sequence which exhibits at least about 80% identity to SEQ ID NO: 1.

10. The cell of claim 8, wherein the E1A TATA box-associated sequence comprises a sequence which exhibits at least about 80% identity to SEQ ID NO: 3.

11. The cell of claim 1, comprising a replication-deficient adenoviral vector having an adenoviral genome deficient in an essential gene function of an early region of the adenoviral genome.

12. The cell of claim 11, wherein the amount of overlap between the cellular genome and the adenoviral genome of the adenoviral vector is such that
(a) the cell produces less than about one replication-competent adenoviral vector for at least about 20 passages after infection with the adenoviral vector,
(b) the cell produces less than about one replication-competent adenoviral vector in a period of about 36 hours post infection,
(c) the cell produces less than about one replication-competent adenoviral vector per 1×10¹⁰-1×10¹³ total viral particles, or
(d) any combination of (a)-(c).

13. The cell of claim 11, wherein the amount of overlap between the cellular genome and the adenoviral genome is insufficient to mediate a recombination event that results in a replication-competent adenoviral vector.

14. The cell of claim 11, wherein there is a region of homology between the cellular genome and the adenoviral genome located 5′ or 3′ to the nucleic acid sequence.

15. The cell of claim 1, wherein the cell is an A549 cell.

16. The cell of claim 1, wherein the cell is a human embryonic kidney cell, a human embryonic retinal cell, a renal leiomyoblastoma, a renal adenocarcinoma cell, a retinal cell, or a small cell lung carcinoma cell.

17. The cell of claim 1, wherein the cell is a non-small cell lung carcinoma cell.

18. A method of propagating a replication-deficient adenoviral vector, which method comprises:
(a) providing a cell comprising a nucleic acid sequence, which upon expression produces a gene product that complements in trans for a deficiency in at least one essential gene function of an adenoviral genome, and which is operatively linked to an expression control sequence, wherein the expression control sequence is upregulated by one or more adenoviral proteins not produced by the nucleic acid sequence,
(b) introducing into the cell a replication-deficient adenoviral vector comprising an adenoviral genome deficient in the at least one essential gene function of the adenoviral genome, and
(c) maintaining the cell to propagate the adenoviral vector.

19. The method of claim 18, wherein the expression control sequence is a chimeric expression control sequence.

20. The method of claim 19, wherein the chimeric expression control sequence comprises at least a functional portion of a CMV immediate early promoter/enhancer region and at least a functional portion of an adenoviral promoter.

21. The method of claim 18, wherein expression of the nucleic acid sequence complements in trans an adenoviral genome comprising deficiencies in at least one essential gene function of the E1 region of the adenoviral genome.

22. The method of claim 21, wherein the cellular genome comprises a nucleic acid sequence comprising an adenoviral E1A coding sequence and an adenoviral E1B coding sequence.

23. The method of claim 22, wherein the cellular genome further comprises a nucleic acid sequence comprising an adenoviral E2 region, E4 region, or both.

24. The method of claim 23, wherein the cellular genome comprises a nucleic acid sequence encoding an E4-ORF6 gene product.

25. The method of claim 20, wherein the chimeric expression control sequence comprises a CMV immediate early enhancer.

26. The method of claim 20, wherein the chimeric expression control sequence comprises an E1A TATA box-associated sequence.

27. The method of claim 25, wherein the CMV immediate early enhancer comprises a sequence which exhibits at least about 80% identity to SEQ ID NO: 1.

28. The method of claim 26, wherein the E1A TATA box-associated sequence comprises a sequence which exhibits at least about 80% identity to SEQ ID NO: 3.

29. The method of claim 18, wherein the adenoviral vector comprises a replication-deficient adenoviral vector having an adenoviral genome deficient in an essential gene function of an early region of the adenoviral genome.

30. The method of claim 18, wherein the amount of overlap between the cellular genome and the adenoviral genome of the adenoviral vector is such that
(a) the cell produces less than about one replication-competent adenoviral vector for at least about 20 passages after infection with the adenoviral vector,
(b) the cell produces less than about one replication-competent adenoviral vector in a period of about 36 hours post infection,
(c) the cell produces less than about one replication-competent adenoviral vector per 1×10¹⁰-1×10¹³ total viral particles, or
(d) any combination of (a)-(c).

31. The method of claim 30, wherein the amount of overlap between the cellular genome and the adenoviral genome is insufficient to mediate a recombination event that results in a replication-competent adenoviral vector.

32. The method of claim 30, wherein there is a region of homology between the cellular genome and the adenoviral genome located 5′ or 3′ to the nucleic acid sequence.

33. The method of claim 18, wherein the cell is an A549 cell.

34. The method of claim 18, wherein the cell is a human embryonic kidney cell, a human embryonic retinal cell, a renal leiomyoblastoma, a renal cell adenocarcinoma, a retinal cell, or a small cell lung carcinoma.

35. The method of claim 18, wherein the cell is a non-small cell lung carcinoma cell.

36. The method of claim 18, wherein the adenoviral vector comprises the one or more adenoviral proteins that upregulate the chimeric expression control sequence.

37. The method of claim 18, wherein the one or more adenoviral proteins that upregulate the chimeric expression control sequence are introduced into the cell independently from the adenoviral vector.
